(19) United States
(12) Patent Application Publication
(10) Pub. No.: US 2020/0107714 A1
(43) Pub. Date: Apr. 9, 2020

(54) SPECULUM DEVICE WITH A DETACHABLE CAMERA AND A METHOD OF USING THEREOF

(71) Applicant: SPECUSIGHT LTD., KIRYAT MOTZKIN (IL)

(72) Inventors: YACKOV BAR-OR, GILON (IL); VICTOR LEVIN, HAIFA (IL)

(21) Appl. No.: 16/500,823

(22) PCT Filed: Feb. 20, 2018
(86) PCT No.: PCT/IL2018/050188

§ 371 Date: Oct. 4, 2019

Related U.S. Application Data
(60) Provisional application No. 62/481,709, filed on Apr. 5, 2017.

(51) Int. Cl. A61B 1/32; A61B 1/04; A61B 1/303; A61B 1/00

(52) U.S. Cl. CPC A61B 1/32; A61B 1/04; A61B 1/00066; A61B 1/00105; A61B 1/303

(57) ABSTRACT

The subject matter discloses a speculum device and a method of using the device. The speculum comprises a body, comprising a lower blade member and an upper blade member, an imaging device for capturing images, and an adapter connected to the body, said adapter is connected to the camera and enables a user of the speculum device to adjust the field of view of the camera relative to the body.

### FIELD OF THE INVENTION

The present invention relates generally to speculum devices, more particularly, it relates to a speculum devices with cameras.

### BACKGROUND OF THE INVENTION

A speculum is a medical tool for investigating body orifices, with a form dependent on the orifice for which it is designed. The best-known speculum is the bivalved vaginal speculum; the two blades are hinged and are “closed” when the speculum is inserted to facilitate its entry and “opened” in its final position where they can be arrested by a screw mechanism, so that the operator is freed from keeping the blades apart.

### SUMMARY OF THE INVENTION

The subject matter discloses a speculum device and method comprises a detachable pan-tilt adapter which can be attached to the speculum, and a guide tube used as a cylindrical pathway to direct the camera in the insertion into the interior of a patient's body. The guide tube may be connected to the pan-tilt adapter in an easy and practical fashion allowing attach and detach the guide tube in a convenient fashion. The pan-tilt adapter can also be attached and detached from the one of the blade members of the speculum. In some cases, the speculum integrated with the camera may be utilized for preforming procedures such as biopsy, video diagnosis, and the like. In some possible embodiments of the present invention, the pan-tilt adapter may comprise spherical joints providing with the ability to perform spherical movements with the camera. Such spherical movements may be tilting, panning, move in or out of the endoscope, and the like. In some cases, the guide tube may also comprise an integrated abutment designed to support the palm of the person operating the medical procedure and allows fine tuning of the camera positioning.

In some cases, the guide tube connected to the pan-tilt adapter may comprise a cone-shaped entrance to allow easy inserting operation of the camera into the guide tube. In some embodiments of the present invention, such guide tube may be made of an expendable material which can be easily replaced. In some cases, the operation of positioning, aiming and/or fixing the camera threaded within the guide tube may be facilitated by an adjustment rod designed to be integrated with the guide tube.

The method disclosed in the subject matter comprises attaching a pan-tilt adapter to a gynecological device, the adapter is attached to a medical imaging device such as a camera. Then, the method comprises inserting the gynecological device into the human vagina, to enable the camera to capture images at the vagina. Then, the method comprises adjusting the camera's angle relative to a blade of the gynecological device, for example by tilting the adapter. Then, the method comprises fixating the camera in the adjusted angle. The adjustment of the camera may be defined by moving the camera's direction from a first direction to a second direction, wherein the second direction has an angle relative to the blade of the speculum device that differs from the angle of the first direction. The method also comprises capturing an image of the patient's body when the camera is in the second position.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-1C show speculum and guide tubes attached to the upper blade member of speculum, according to exemplary embodiments of the present invention;

FIG. 2 shows speculums and guide tubes attached to the lower blade member of a speculum, according to exemplary embodiments of the present invention;

FIG. 3 shows a schematic representation of two possible camera types which can be used in speculum operations, according to exemplary embodiments of the present invention;

FIGS. 4A-4B show two possible cameras designed to be inserted into guide tubes of two possible speculums, according to exemplary embodiments of the present invention;

FIG. 5 shows a possible camera threaded into a guide tube mounted onto the lower blade ember of a possible speculums, according to exemplary embodiments of the present invention;

FIG. 6 shows a possible camera threaded into a guide tube mounted on the upper blade member of a possible speculums, according to exemplary embodiments of the present invention;

FIGS. 7A-7B demonstrate two cross-section views of a possible PTF adapter designed to be connected to a speculum and host a camera, according to exemplary embodiments of the present invention;

FIGS. 8A-8H demonstrate cross-section views of a possible PTF adapter designed to be connected to a speculum and host a camera, according to exemplary embodiments of the present invention;

FIGS. 9A-9B show two speculums with cameras, wherein each a camera is pointing to a different direction, according to exemplary embodiments of the present invention;

FIG. 10A shows a camera with a bending section inserted into a guide tube mounted on a speculum via PTF adapter, according to exemplary embodiments of the present invention;

FIG. 10B shows a speculum with a camera inserted into a guide tube mounted on a PTF adapter, according to exemplary embodiments of the present invention;

FIG. 11 shows a speculum with a camera inserted into a guide tube mounted on a PTF adapter, according to exemplary embodiments of the present invention;

FIG. 12 shows a guide tube with an abutment designed to control the guide tube and the camera pointing direction during medical procedures with a speculum, according to exemplary embodiments of the present invention;

FIG. 13 shows a camera with a sleeve designed to be connected to a speculum by using a camera adapter, according to exemplary embodiments of the present invention;

FIGS. 14A-14C show a 3 step process to fasten a camera to a camera adapter, according to exemplary embodiment of the present invention;

FIG. 15 shows a camera with a sleeve attached to a speculum by using a camera adapter, according to exemplary embodiments of the present invention;

FIG. 16 shows a camera which can be attached to a speculum by using a camera adapter, according to exemplary embodiments of the present invention;

FIG. 17 shows a camera which can be attached to a speculum by using a camera adapter which allows the necessary leeway to tilt up the camera and reach an acute position upwards, according to exemplary embodiments of the present invention;

FIG. 18A shows a camera with a sleeve attached to a connecting platform which allows to pan the camera in the horizontal dimension, according to exemplary embodiments of the present invention;

FIG. 18B demonstrates the operation changing the camera of an endoscope in the horizontal dimension by using a pan wheel and a steering wheel, according to exemplary embodiments of the present invention;

FIGS. 19A-19B show a flexible gripper designed to connect a camera to a speculum and provide the ability to change the direction to which the camera is pointing to, according to exemplary embodiments of the present invention; and,

FIGS. 20A-20B show a flexible gripper and a locking mechanism of a speculum to a specific direction, according to exemplary embodiments of the present invention.

### DETAILED DESCRIPTION OF THE INVENTION

The present invention discloses a detachable pan-tilt and fixation adapter which can be utilized to mount an imaging medical device on a speculum. The detachable Pan-Tilt, and Fixation adapter, defined herein as PTF adapter, can be utilized for inserting, positioning, adjusting, and/or aiming the imaging medical device attached to the speculum. In some cases, inserting the imaging medical device may be facilitated by a guide tube attached to the PTF adapter in order to direct the insertion act of the imaging medical device to be situated between the two speculum blade members. In some embodiments of the present invention, the operation of positioning, aiming and/or fixing the imaging medical device threaded within the guide tube may be facilitated by an adjustment rod designed to be integrated with the guide tube.

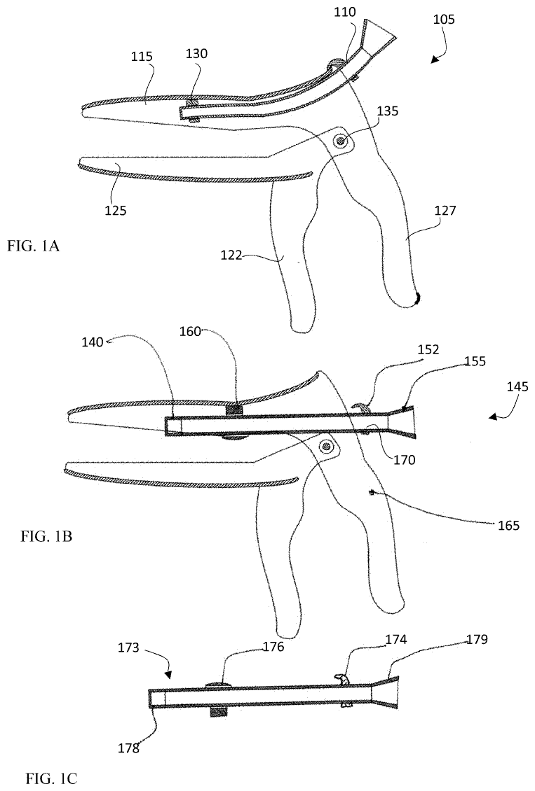

FIGS. 1A-1C show speculum and guide tubes attached to the upper blade member of speculum, according to exemplary embodiments of the present invention. FIG. 1A shows a speculum 105 comprising an upper blade member 115 and a lower blade member 125 configured to widen the vaginal walls of a patient. The blades members are pivoted on hinge 135 structured at their ends so their handles can function as operating levers. Hence, rear handle 122 which can function as an operating lever for the lower blade 125, and front handle 127 which can function as an operating lever for the upper blade 115. Speculum 105 also comprises a curved guide tube 110 designed to direct the insertion of an imaging device into a cavity in a body. For example, in a medical procedure with a speculum a person can utilize the speculum 105 to widen the vaginal walls of a patient. Then, the camera can be pushed and smoothly inserted through the curved guide tube 110 till the view required for that specific medical procedure is provided. In some cases, the curved guide tube 110 may be made of a rigid material along its entire length. The curved guide tube 110 can be attached to the upper blade member 115 of speculum 105 via PTF adapter 130. In some cases, the PTF adapter 130 may have a spherical form which allows the person, utilizing the imaging device, to aim and adjust the position of the imaging device by making panning operations, tilting operations or rotate the camera in order to achieve optimal view with the camera.

FIG. 1B also shows a speculum 145 functioning and designed as aforementioned. Speculum 145 comprises a straight guide tube 170 designed to guide the insertion act of an imaging device such as camera. The straight guide tube 170 comprises a cone shaped entrance 155 designed to facilitate the insertion of the camera into the straight tube 170. For example, a person operates an camera in a medical procedure can insert the camera to the straight guide tube 170 via aiming the camera to a relatively wide entrance such as cone shaped entrance 155 provides. Once the camera has been inserted into the cone shaped entrance 155, the camera can be smoothly threaded through the entire length of straight guide tube 170. The straight guide tube 170 may be mounted to the speculum 145 via PTF adapter 160. The user who operates the camera may utilize the PTF adapter 160 to change the position or improve the sight of the camera as described above. The user who operates the speculum 145 may also be able to utilize the handle 165 to situate the camera in an optimal view. For example, a user who operate the speculum 145 may change the opening width between the blade members of the speculum and thereby change the position of the camera inserted the cavity of the body. Straight guide tube 170 also comprises pipe head 140 designed to host the camera and to hold the camera in a stable and firmed state. The speculum 145 also comprise a lock 152 utilized for fixation of the two blade members of the speculum 145, so the distance between the blade members is fixed and remains constant. In some cases, the lock 152 can be a plunger lock, trigger latch, elongated curved latch, and the like.

FIG. 1C also shows a straight guide tube 173 designed to be mounted on a speculum via PTF adapter 176. The PTF adapter 176 may be a detachable adapter designed to be attached and/or detached from a speculum. In some cases, the PTF adapter 176 may be attached to the speculum via materials such as, vacuum sucker, click joints, magnetic locks, glue, stickers, adhesive material, screws, and the like. In some other cases, the PTF adapter 176 may be attached to the speculum via dedicated tiny rails placed in one of the speculum's blades. Such tiny rails may be designed to host the PTF adapter and provide the infrastructure which fixes the PTF adapter and thereby the camera movement.

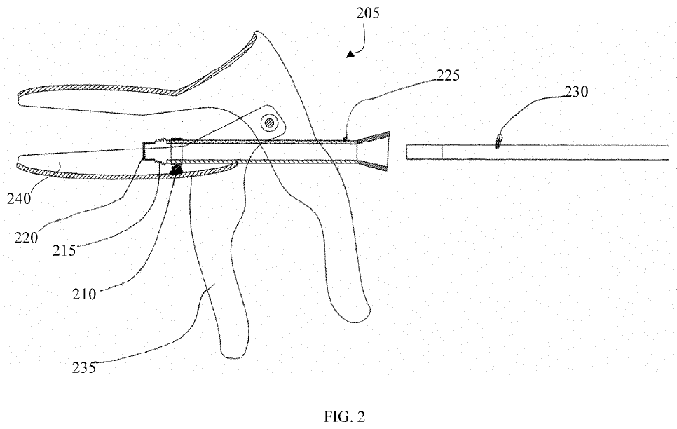

FIG. 2 shows speculums and guide tubes attached to the lower blade member of a speculum, according to exemplary embodiments of the present invention. FIG. 2 demonstrates a speculum 205 comprising a PTF adapter 210 attached to the lower blade member 240. The speculum 205 also comprises a guide tube 225 attached to the PTF adapter 210 and designed to guide the insertion act of an imaging device such as camera 230. Guide tube 225 comprises a narrowed head 215 designed to fasten the camera 230 and provide a firmed host which stabilizes the camera 230 via providing the minimum possible leeway within the guide tube 225. As a result of the cone head 215 design, the movement of the camera 230 can be achieved by moving the guide tube 225. The guide tube 225 also comprises a round aperture 220 used to provide the field of view of the imaging device such as camera 230. The speculum 205 also comprises a handle 235 which can function as operating levers of lower blade member 240. In some cases, the handle 235 can be used to change the position of lower blade member 240 and thereby to change the position of the camera 230 threaded in guide tube 225. In some cases, changes in the position of the lower blade member 240 can cause changes in the position of the camera 230, and by that cause to changes in the field of view captured by the camera 230. In such cases, the changes can be corrected by changing the position of guide tube 225 connected to PTF adapter 210.

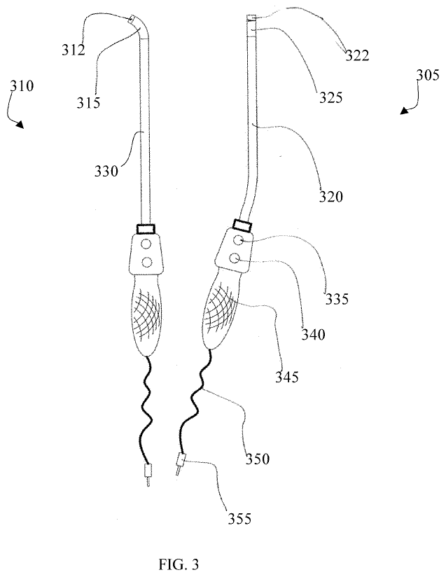

FIG. 3 shows a schematic representation of two possible endoscope types which can be used in speculum operations, according to exemplary embodiments of the present invention. FIG. 3 shows two endoscopy devices, endoscope 310 and endoscope 305. Both endoscopy devices, 305 and 310 can be inserted to the guide tube and be utilized in the course of speculum operations. Endoscopy device 310 comprises an insertion tube 320 which can be threaded into a guide tube attached to a PTF adapter. The insertion tube 320 may be a part of diverse type of endoscopy devices which can be inserted into the guide tube and be utilized in the course of the speculum operation. Endoscopy device 305 also comprises a straight section 325 designed to deliver the endoscope components to the end of the endoscope. Such endoscope components may be endoscope cords, wires to carry electricity and light, air pipe, water pipe and the like. The endoscope 305 also comprises an endoscope tip 322 located at the end of the straight section 325 and utilized to carry the endoscope components, composition and wires to the edge of the endoscope. The endoscope tip 322 may comprise, lenses, light source, insufflation nozzle, suction channel, camera, and the like.

Endoscopy device 305 also comprises valve 340, and 335 which are endoscope valves utilized in the standard procedure of the endoscope. Such valve can be a suction valve, a water valve, air valve, and the like. In some embodiments of the present invention, the endoscopy device 305 may not comprise any valve such as valve 340, and/or valve 335. In some cases, the endoscopy device 305 may comprise more than two valves. The endoscopy device 305 may also comprise additional entries such as a biopsy entry, instrument channel and the like. Endoscopy device 305 also comprises a universal cord 350 designed to carry the endoscope wires and channels. Such wire and channels may be light source, a camera connection, suction channel, and the like. The universal cord 350 also comprises endoscope plug 355. In some cases, the endoscope plug 355 may be used to connect the endoscopy device 305 to a power source. In some other cases, the endoscope plug 355 may also comprise a light source connection, video cable connection, pump connections, and the like.

Similarity, the endoscopy device 310 may also comprise insertion tube 330 which can be threaded into a guide tube of the speculum, in similar manner as insertion tube 320. Endoscopy device 310 also comprises a bending section 315 designed to deliver the endoscope components to the end of the endoscope. In some cases, in accordance with the protocol of the medical procedures performed by the speculum, a bending section such as bending section 315 may be utilized. In such cases, the guide tube integrated with the PTF adapter can allow the insertion of the endoscopy devices with bending sections, in similar manner as it is done with the straight section 325. For example, an endoscopy device 305 can be used in a medical procedure performed with a speculum. Then, the person who operates the medical procedure may decide to remove the endoscopy device 305 and start utilize an endoscope with a bending section such as endoscopy device 310. The person who operates the medical procedure may be able to remove the endoscopy device 305 and insert the endoscopy device 310 in a seamless and easy fashion. Endoscopy device 310 also comprises an endoscope tip 322 located at the end of the bending section 315 and utilized to carry the endoscope components in similar manner as endoscope tip 312 aforementioned.

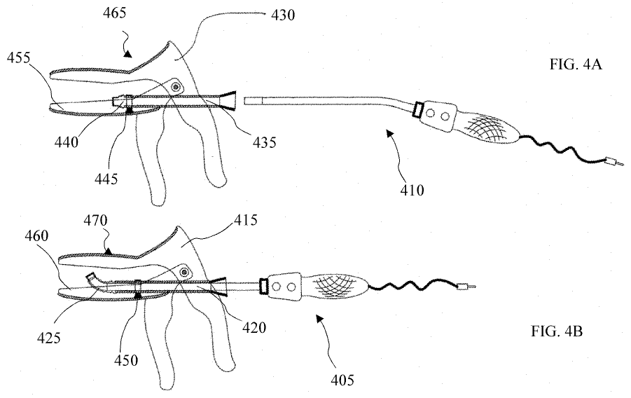

FIGS. 4A-4B show two possible endoscopy devices designed to be inserted into guide tubes of two possible speculums, according to exemplary embodiments of the present invention. FIG. 4A shows endoscopy device 410 with a straight section 440 designed to be inserted into the guide tube 435. Guide tube 435 is mounted onto speculum 465 via PTF adapter 445 which may be connected to the lower blade member 455. In some cases, the PTF adapter 445 may be connected to the upper blade member 430 of the speculum 465, and by that, the endoscopy device 410 may be inserted into the guide tube 435 as it was mounted to the upper blade member 430.

FIG. 4B also shows endoscopy device 405 with a bending section 425 designed to be inserted into guide tube 420. Guide tube 420 is mounted onto the speculum 470 via PTF adapter 450 which may be connected to the lower blade member 460. In some cases, the PTF adapter 450 may be connected to the upper blade member 415 of the speculum 405, and by that, the endoscopy device 405 may be inserted into the guide tube 420 and thus mounted to the upper blade member 415.

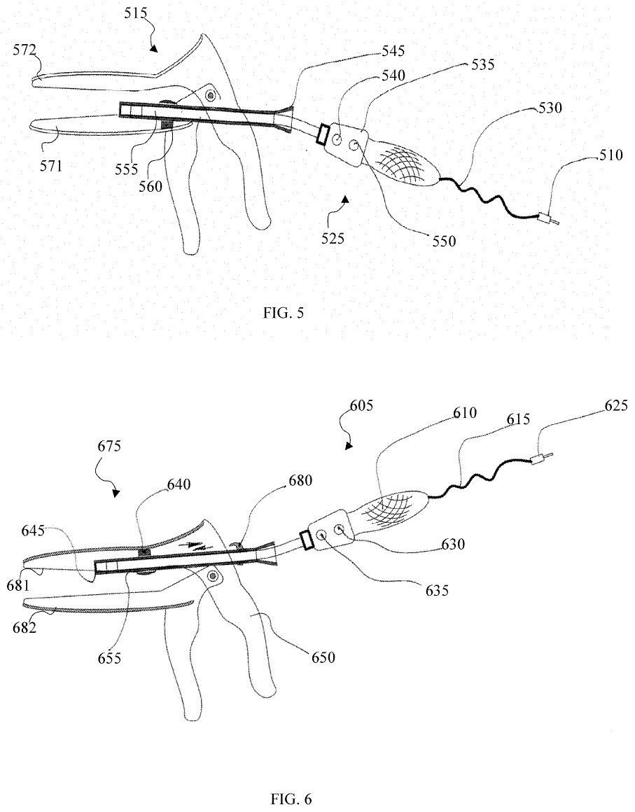

FIG. 5 shows a camera threaded into a guide tube mounted onto the lower blade ember of a possible speculums, according to exemplary embodiments of the present invention. FIG. 5 shows a camera 525 threaded into a guide tube 555. In this possible embodiment of the present invention, the guide tube 555 can be mounted onto a speculum 515 via PTF adapter 560 which can be connected to the lower blade member 571 of speculum 515. In some cases, the PTF adapter 560 may be connected to the upper blade member 572 of speculum 515 and the guide tube 555 may be mounted onto the upper blade member 572 and thereby allow the camera to be attached to the upper blade member 572. The guide tube 555 comprises a cone shaped entrance 545 designed to facilitate the insertion act of the camera 525 into the guide tube 555.

The speculum device 525 threated into a guide tube 555 comprises two valves, a valve 540 and valve 550 designed to facilitate the speculum operation. The speculum device 525 also comprises a cable 530 which can deliver electric power, light illumination, and other resources related to the medical procedures. In some cases, the cable 530 can be a universal cord. The speculum device 525 also comprises a plug 510 designed to deliver electric power, light source, and the like. The speculum device 525 also comprises a handle 535 situated at the outer portion of the speculum device 525 and can be utilized to push the speculum device 525 into the guide tube 555. The handle 535 can also be utilized to aim the camera, and adjust the position of the speculum device 525 within the guide tube 555.

FIG. 6 shows an endoscopy device threaded into a guide tube mounted on the upper blade member of a speculum, according to exemplary embodiments of the present invention. FIG. 6 shows an endoscopy device 605 threaded into a guide tube 655. The guide tube 655 is mounted onto speculum 675 via PTF adapter 640 connected to the upper blade member 681 of speculum 675. In some cases, the PTF adapter 640 may be attached to the lower blade member 682 of the speculum 605 and thus, the guide tube 655 may be mounted to the lower blade member 682. The endoscopy device 605 which can be threated into a guide tube 655 comprises two valves, a valve 630 and valve 635 designed to facilitate the endoscope operation as aforementioned. The guide tube 655 may also comprise an endoscope tip 645 located at the end of the guide tube 655 and utilized to carry the endoscope component as aforementioned.

The endoscopy device 605 also comprises a cable 615 and a plug 625 which may function as aforementioned. The endoscopy device 605 also comprises a handle 610 situated at the outer portion of the endoscopy device 605 and can be utilized to adjust the position of the endoscopy device 605 in order to aim it toward specific objects, as part of a medical procedure. In some cases, the speculum handle 650 may also be used to change the position of the endoscopy device 605, or change the direction of the endoscopy device 605. In medical procedures, the lock 680 may be used to fix the distance between the two blade members of the speculum 675.

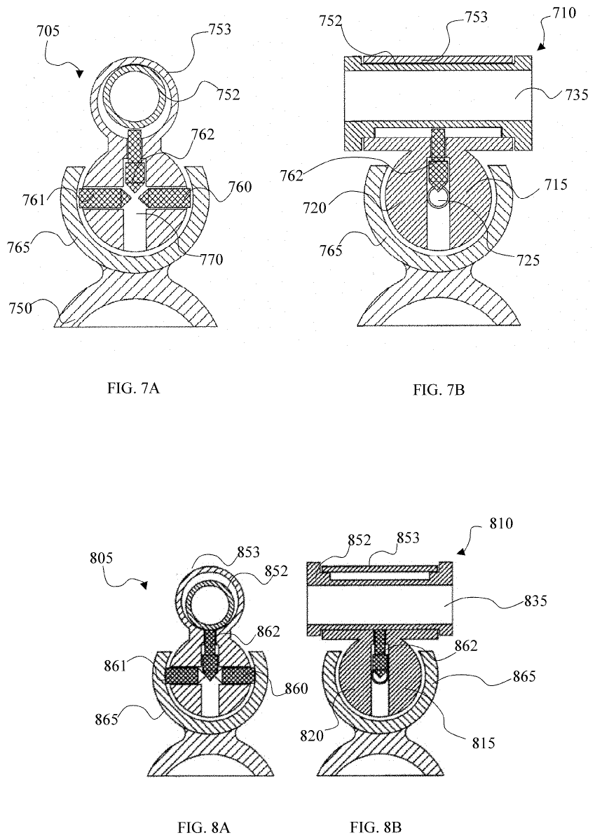

FIGS. 7A-7B demonstrate two cross-section views of a possible PTF adapter designed to be connected to a speculum and host an endoscopy device, according to exemplary embodiments of the present invention. FIG. 7A shows a cross-section view of PTF adapter 705. The cross-section view of PTF adapter 705 is perpendicular to the inserting direction the endoscope, namely, an endoscope may be inserted in via the eccentric socket 752. The PTF adapter 705 comprises rods 761 and 760 designed to provide an outer pressure which increases the friction against the round wall 765. Rod 761 and 760 may also provide a pressure toward rod 762 pushed upwards against the adapter's round-shaped casing 753 and thereby can lock the movement of the eccentric socket 752. In some embodiments of the present invention, springs may be added to the rods 761, 762 and 760 to increase the friction against the round wall 765, created by the pressure of rods 760, 761 and 762. The cross-section view of PTF adapter 705 also shows the adapter basis 750 which may be used to connect the adapter to the speculum.

FIG. 7B also shows a cross-section view of PTF adapter 710. The cross-section view of PTF adapter 710 is in parallel to the inserting direction of the endoscope. The cross-section view of PTF adapter 710 shows the path 735 through which the endoscope may be inserted into a cavity of the body. The cross-section view of PTF adapter 710 also exposes the eccentric socket 752 shown as upwardly pressed against the adapter's round-shaped casing 753 and thereby can lock the movement of the eccentric socket 752. The cross-section view of PTF adapter 710 also shows the spherical joints 720, and 715 which comprise the rods 760, 761 and 762 which pressure the spherical joints 720 and 715 toward the round wall 765 and by that block the movement of the endoscope which may be threated into the eccentric socket 752 through path 735.

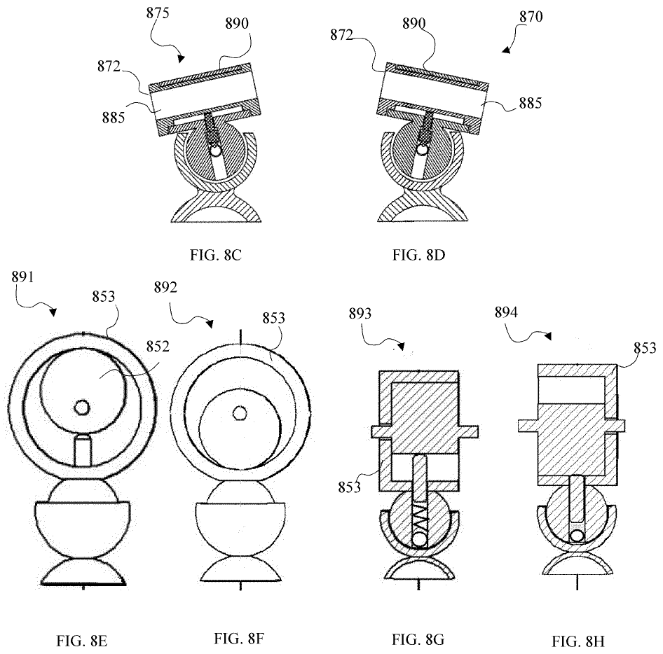

FIGS. 8A-8H demonstrate two cross-section views of a possible PTF adapter designed to be connected to a speculum and host an endoscopy device, according to exemplary embodiments of the present invention. FIG. 8A shows a cross-section view of PTF adapter 805. The cross-section view of PTF adapter 805 is perpendicular to the inserting direction the endoscope, that is to say, an endoscope may be inserted in via the eccentric socket 852. The PTF adapter comprises rods 861 and 860 designed to provide an outer pressure which increases the friction against the round wall 865. Rods 861 and 860 may also provide a pressure toward rod 862 pushed downwards against the adapter's round-shaped casing 853 and thereby can lock the movement of the eccentric socket 852.

FIGS. 8A-8B also show a cross-section view of PTF adapter 810. The cross-section view of PTF adapter 810 is in parallel to the inserting direction of the endoscope. The cross-section view of PTF adapter 810 shows the path 835 through which the endoscope may be inserted into a cavity of a body. The cross-section view of PTF adapter 810 also shows the eccentric socket 852 shown as downwardly pressed against the adapter's round-shaped casing 853 and thereby can lock the movement of the eccentric socket 852. The cross-section view of PTF adapter 810 also shows the spherical joints 820 and 815 which comprise the rods 860, 861 and 862 (not shown) which pressure the spherical joints 820 and 815 toward the round wall 865 and by that block the movement of the endoscope which may be threated into the eccentric socket 852 through path 835.

FIGS. 8C-8D demonstrate two cross-section views of two possible positions of a PTF adapter designed to be connected to a speculum and host an endoscopy device, according to exemplary embodiments of the present invention. FIGS. 8C-8D show a cross-section view of PTF adapter 870. The cross-section view of PTF adapter 870 is in parallel to the inserting direction the endoscope. The cross-section view of PTF adapter 870 shows the path 885 through which the endoscope may be inserting into a cavity of a body. The cross-section view of PTF adapter 870 also exposes the eccentric socket 872 which shown as upwardly pressed against the adapter's round-shaped casing 890 and thereby can lock the movement of the eccentric socket 852. The cross-section view of PTF adapter 870 shows a possible position of the PTF adapter 870 which points the eccentric socket 872 tilted upward. Thus, an endoscopy device which may be inserted into the eccentric socket 872 directed upwards. In some cases, the a PTF adapter shown in cross-section view 870 may be fixed without a leeway allowing to move freely, rightwards, leftwards or rotate, namely that the endoscope can preserve field of view captured by the endoscope camera stable and fixed.

FIG. 8C also shows a cross-section view of PTF adapter 875. The cross-section view of PTF adapter 875 is in parallel to the inserting direction the endoscope. The cross-section view of PTF adapter 875 shows the path 885 through which the endoscope may be inserting into a cavity of a body. The cross-section view of PTF adapter 875 also exposes the eccentric socket 872 shown as upwardly pressed against the adapter's round-shaped casing 890 and thereby can lock the movement of the eccentric socket 852. The PTF adapter can allow fixing the camera in respect to the speculum. The cross-section view of PTF adapter 875 shows a possible position of the PTF adapter 875 which points the eccentric socket 872 tilted downward. Thus, an endoscopy device which may be inserted into the eccentric socket 872 may be directed downwards. In some cases, the [a] PTF adapter shown in cross-section view 870 may be fixed without a leeway allowing to move freely, rightwards, leftwards or rotate, namely that the endoscope can preserve the field of view captured by the endoscope camera stable and fixed. In some cases, the PTF adapter may be pointed leftwards and/or rightwards in the same manner as it is being done to point the adapter upwards or downwards.

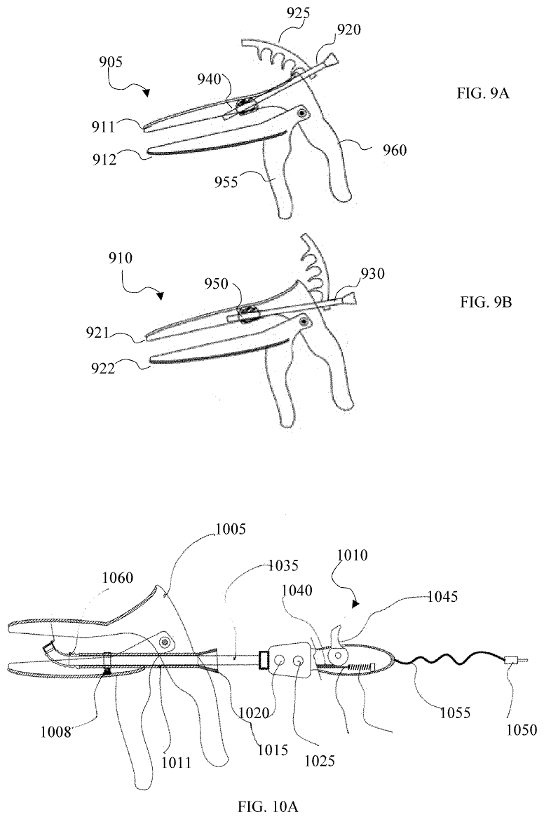

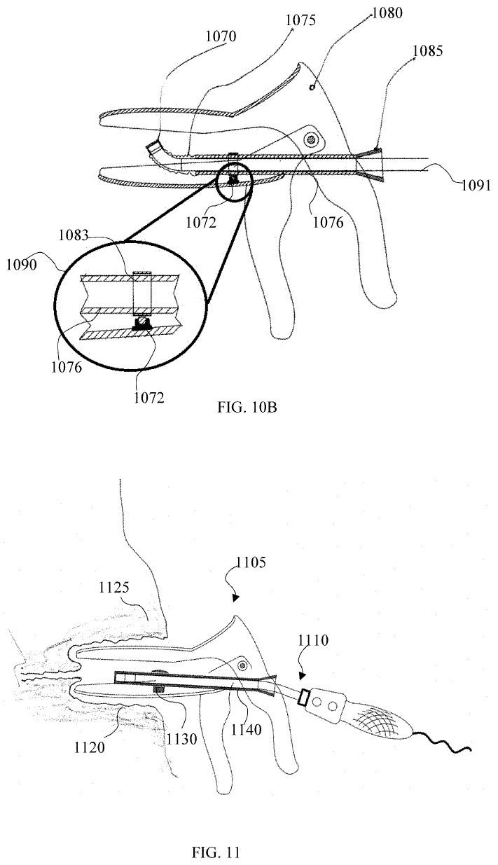

FIGS. 9A-9B show two speculums with endoscopy devices, wherein each endoscopy devices is pointing to a different direction, according to exemplary embodiments of the present invention. FIG. 9A shows a speculum 905 comprises an upper blade member 911 and lower blade member 912 designed to widen the vaginal walls of a patient. The speculum 905 comprises a latch 925 comprising latching teeth on the side facing the speculum blade. The latch 925 may be used to establish and maintain a fixed distance between the upper blade member 911 and the lower blade member 912 during a medical procedure facilitated by the speculum 905. For example, a person operating the speculum 905 may widen the vaginal walls of a patient. Then, said person may utilize the latch 925 to set a fixed distance between the two speculum blades in order to keep a fixed size of the orifice created between the vaginal walls of the patient. In some cases, the integrated latch 925 may be replaced by a screw, a plunger lock, and the like.

Speculum 905 also comprises a guide tube 920 mounted to the upper blade member 911 by PTF adapter 940. The PTF adapter 940 can allow a person to change the direction of the guide tube 920, to tilt or pan the guide tube 920, and the like. The guide tube 920 is set to be tilted downwards and point toward the edge of the lower blade member 912. Guide tube 920 may be used to insert an endoscopy device to a cavity created by the speculum 905. In some cases, the latch 925 can be unlocked so additional adjustments can be made by changing by utilizing handle 955 and handle 960 in order to change the distance between the upper blade member 911 and the lower blade member 912.

FIG. 9B also shows a speculum 910 comprises an upper blade member 921 and lower blade member 922 designed to widen the vaginal walls of a patient. Speculum 910 also comprises a guide tube 930 mounted to the upper blade member 921 by PTF adapter 950. The guide tube 930 can be set to be tilted upwards and be aligned with the upper blade member 922. Guide tube 930 may be used to insert an endoscopy device to the cavity created by the speculum 910.

FIG. 10A shows an endoscopy device with a bending section inserted into a guide tube mounted on a speculum via PTF adapter, according to exemplary embodiments of the present invention. FIG. 10A shows an endoscopy device 1010 threaded into a guide tube 1011. Guide tube 1011 comprises a cone shaped entrance 1015 and can be mounted onto speculum 1005 via PTF adapter 1008.

The endoscopy device 1010 comprises two valves, valve 1020 and valve 1025 designed to facilitate the endoscope operation as aforementioned. The endoscopy device 1010 also comprises a cable 1055, which in some cases can be a universal cord. The endoscopy device 1010 also comprises a plug 1050, as disclosed above. The endoscopy device 1010 also comprises a bending section 1060 and an insertion section 1035 which can be threaded into the guide tube 1011 via cone shaped entrance 1015.

FIG. 10B shows a speculum with an endoscopy device inserted into a guide tube mounted on a PTF adapter, according to exemplary embodiments of the present invention. FIG. 10B shows an endoscopy device 1091 threaded into a guide tube 1076 via cone shaped entrance 1085. The guide tube 1076 is mounted on speculum 1080 via PTF adapter 1072. The endoscopy device 1091 comprises a bending section 1075 with endoscope tip 1070 utilized for the medical operations as aforementioned. FIG. 10B also shows a magnified illustration 1090 which demonstrates the PTF adapter 1072. Magnified illustration 1090 demonstrates the PTF adapter 1072 connected on the top surface 1081 of the lower blade member 1087 of speculum 1080. In some embodiments of the present intention, the PTF adapter may comprise an annular gripper such as annular gripper 1083. The magnified illustration 1090 also demonstrates the guide tube 1076 inserted the annular gripper 1083 which can grip the guide tube 1076 and allow diverse optional movements with the endoscopy device 1091 such as panning, tilting, rotating, and the like.

FIG. 11 shows a speculum with an endoscopy device inserted to a body cavity, according to exemplary embodiments of the present invention. FIG. 11 shows a camera 1110 threated to guide tube 1140 mounted on speculum 1105 via PTF adapter 1130. The speculum 1105 may be used to widen the cervical cavity exists between cervical wall 1120 and cervical wall 112. In some cases, the camera 1110 may be utilized as part of a medical procedure performed with the speculum 1105. In some other cases, the usage of the speculum 1105 may be to support and enable an endoscopy operation or a medical procedure which merely utilized by the camera 1110. A user which operates the camera 1110 may be able to pan, tilt, and rotate the guide tube 1140 and thereby change the position of the camera 1110, accordingly. For example, in case a user may change the position of the guide tube 1140, the camera 1110 may also change the direction to which the endoscope 1110 is pointing. In some case, changes with the view of the camera 1110 may be achieved by changing the endoscope positon within the guide tube 1140. For example, a person operates the camera 1110 may push the camera 1110 inside or pull it outside, without changing the position of the guide tube 1140.

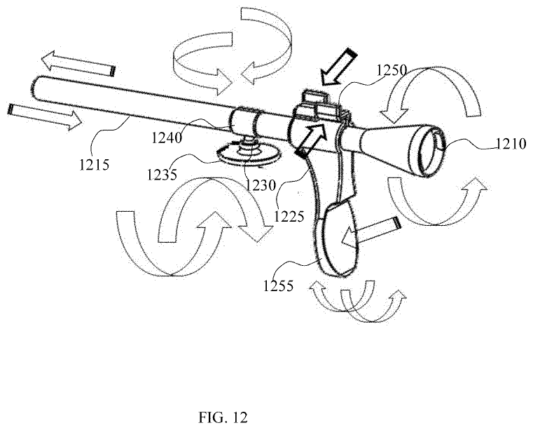

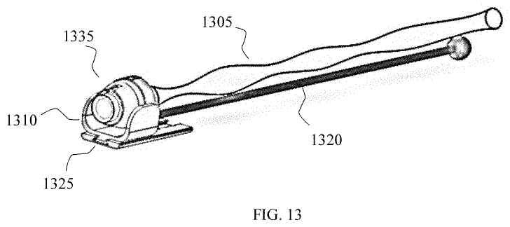

FIG. 12 shows a guide tube with an abutment designed to control the guide tube and the endoscope pointing direction during medical procedures with a speculum, according to exemplary embodiments of the present invention. FIG. 12 shows a guide tube 1215 with cone-shaped entrance 1210 designed as aforementioned. The guide tube 1215 may be used for inserting an endoscopy device into a cavity in a body. The guide tube 1215 may have an abutment 1225 mounted between the cone shaped entrance 1210 and the PTF adapter 1240. The abutment 1225 may have a palm holder 1255 which can be utilized by the person who operate the medical procedure in order to rotate, pan, tilt, push in, and push out the guide tube 1215 and thereby to change the direction to which the camera is pointing. For example, a person can lay the palm on the palm holder 1255, press with the palm on the palm holder 1255 and then rotate to the left, to the right, or to push it in the guide tube 1215. Palm holder 1255 also comprises a clamp 1250 utilized to fasten the palm holder 1255 to the guide tube 1215.

In possible embodiments of the present invention, the PTF adapter 1240 can comprise a spherical joint 1230 which may enable the guide tube 1215 to move in spherical movements. The PTF adapter 1240 may also comprise an adapter base 1235 which can be attached to a wall of a medical device. For example, the adapter base 1235 may be attached to one of the blade members of a speculum.

FIG. 13 shows a camera with a sleeve designed to be connected to a speculum by using a camera adapter, according to exemplary embodiments of the present invention. FIG. 13 shows a camera 1335 connected to adapter 1310. The adapter 1310 may be designed to accommodate the camera 1335 and fix it in a stabilized fashion. Camera adapter 1310 also comprises a connecting platform 1325 designed to be connected to a medical device such as a speculum. In some cases, an adhesive material can be used to connect the connecting platform 1325 to the medical device. In some other cases, screws, clasps, magnetic surface, and the like may be used to connect the connecting platform 1325 to the medical device. FIG. 13 also shows an expendable sleeve 1305 utilized to cover cables and wires the camera 1335 may have. For example, a video cable or a power cable may be connected to the camera 1335. Thus, the cables and the wires can be inserted into the expendable sleeve 1305. FIG. 13 also shows a control rod 1320 designed to control the movements of the camera 1335. For example, in case a user wishes to change the position of the camera 1335, or change the direction to which camera 1335 is pointing, the user may utilize the control rod 1320 to rotate, tilt or pan the camera 1335. The control rod 1320 may be designed to reach out of the body cavity to which the medical device is inserted, and provide the user with a convenient mode of operation.

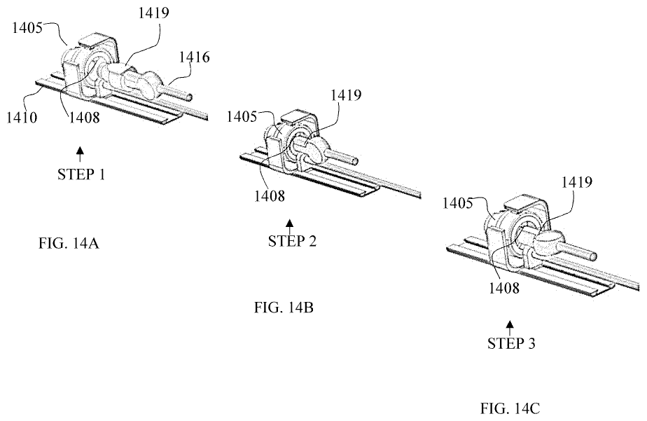

FIGS. 14A-14C show a 3 step process to fasten a camera to a camera adapter, according to exemplary embodiment of the present invention. FIGS. 14A-14C show steps 1, 2, and 3 for fastening the camera 1419 into camera adapter 1405. In FIG. 14A, the camera 1419 is situated outside of the camera adapter 1405. Camera 1419 also comprises a cable 1416 which in some cases, can be a power cable. In some other cases, additional cables may be connected to the camera 1419, such as a cable utilized as a light source, a video cable for the camera 1419, and the like. The camera adapter 1405 also comprises a locking tooth 1408 which can be used to lock the camera 1419 inside the camera adapter 1405. In some cases, the camera 1419 may comprise a slot or a socket to accommodate the locking tooth 1408. The camera adapter 1405 also comprise a connecting platform 1410 as aforementioned.

FIG. 14B shows the camera 1419 inserted into the camera adapter 1405. In some cases, the camera 1419 may be required to be inserted into the camera adapter 1405 in a specific angle so it fits into the adapter in a specific position. In some cases, a specific entrance angle of the camera 1419 may need to be provided in order to accommodate the locking tooth 1408.

FIG. 14C shows a camera 1419 inserted into a camera adapter 1405 after rotating the camera to the right in order to lock the camera 1419 to the camera adapter 1405. In some cases, such a lock may be provided by the locking tooth 1408. In some embodiments of the present invention, more than one locking tooth may be placed in the camera adapter 1405. In such cases, more than one rotation may be used. For example, the camera may need to be rotated to the right, and then after locking one tooth, to be rotated to the left or to the right.

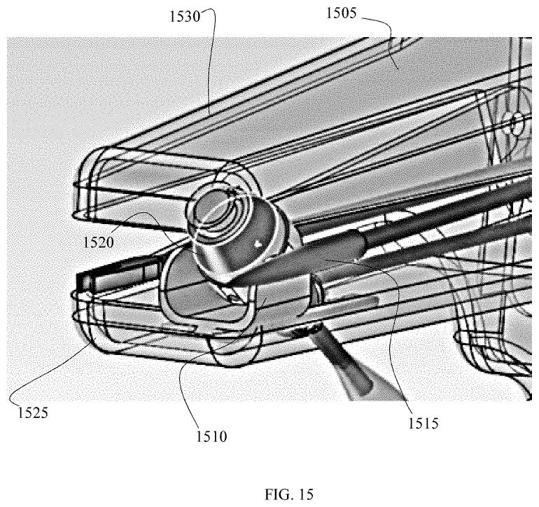

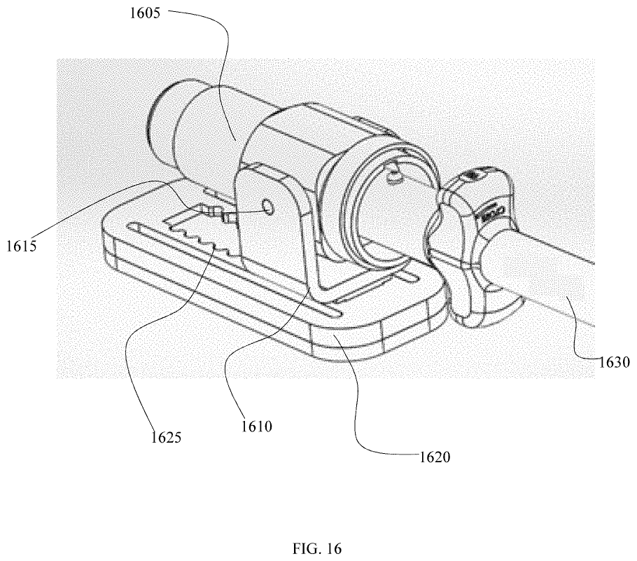

FIG. 15 shows a camera with a sleeve attached to a speculum by using a camera adapter, according to exemplary embodiments of the present invention. FIG. 15 shows a camera 1520 connected to camera adapter 1510 and attached to speculum 1505. The camera adapter 1510 may be designed to accommodate the camera 1520 and fix it in a stabilized fashion. The camera adapter 1510 may also be designed to provide a tilt functionality to the camera 1520 and reach an obtuse angle up to 90 degree upward or, in some cases, downwards. Thus, the camera 1520 can be tilted upwards or downwards, be positioned in an obtuse angle and allow the user of the speculum to utilize the speculum in a relatively narrow opening between the speculum blade member 1530 and speculum blade member 1525. For example, the speculum user can insert the speculum into a vaginal cavity and fix the two-speculum member blades 1525 and 1530 in a narrow opening and then tilt the camera 1520 fastened on camera adapter 1510 up to 90 degree in order to capture a broaden field of view. In some cases, the camera 1520 may be connected to camera adapter 1510 with a hinge (not shown) in order to provide a rotation functionality to the camera 1520.

Camera 1520 may also be connected to a control rod 1515 designed to control the camera position. Rod 1515 can be pulled and pushed in and out by a user and thereby to change the camera position. In some cases, the control rod 1515 may be utilized during the medical operation in order to adjust the camera field of view. For example, in a specific medical procedure, the speculum user may push in or pull out the control rod 1515 and thereby change the angle of the camera. Once the camera 1520 is repositioned and the field of view is adjusted, the speculum user can proceed with the medical operation.

FIG. 16 shows a camera which can be attached to a speculum by using a camera adapter, according to exemplary embodiments of the present invention. FIG. 16 shows a camera 1605 connected to camera adapter 1610 designed to accommodate the camera 1605 and fix it in a stabilized fashion. Camera adapter 1610 can comprise a connecting platform 1620 designed to be connected to a medical device such as speculum. Camera adapter 1610 may also comprise a jagged aperture 1625 configured to fasten the camera 1605 in a specific location. In some cases, the jagged aperture 1625 may be utilize to change the location of the camera 1605 without removing or changing the location of the camera adapter 1610. For example, a user of the camera 1605 requires to move the camera 1605 forwards or backwards, after the connecting platform 1620 is connected to a medical device such as a speculum. The user can utilize the jagged aperture 1625 to relocate the camera in a different position by pushing the camera 1605 forwards or backwards along the jagged aperture 1625. In some cases, the camera adapter 1610 may comprise an additional mechanism to lock the camera 1605 in a specific location along the jagged aperture 1625. Such additional mechanism may be a screw, a clasp, and the like.

Camera 1605 may also comprise a sleeve 1630 which in some cases can be an expendable sleeve. In some cases, sleeve 1630 may be a rigid sleeve which can be utilize to control the location of the camera 1605. For example, during a medical procedure a user of a speculum can utilize the sleeve 1630 to push the camera in or out, along the jagged aperture 1625 and thus, to change the location of the camera 1605. In some embodiments of the present invention a user of the camera 1605 may utilize the sleeve 1630 to lock the position of the camera. For example, a user may hold and rotate the sleeve 1630 clockwise, or in some cases counterclockwise in order to lock and fix the camera 1605 in a stabilized fashion at a specific location along the jagged aperture 1625.

Camera adapter 1610 also comprise a hinge 1615 allowing to pivot and control the angle of the camera 1605. In some cases, pivoting the camera 1605 may take place during the medical operation in order to adjust the camera field of view. For example, in a specific medical procedure, the speculum user may pivot the camera 1605 around the hinge 1615 and thereby tilt up or tilt down the camera 1605. Once the angle of the camera 1605 is changed and the field of view is adjusted, the speculum user can proceed with the medical operation. In some cases, such changes in the angle of the camera 1605 may be done via a dedicated rod utilized to reach the camera 1605 from the outside of the speculum. In some other cases, the user of the camera may change the position of the camera 1605 along the jagged aperture 1625 and also change the camera 1605 position upwards or downwards and thereby change the field of view of camera 1605.

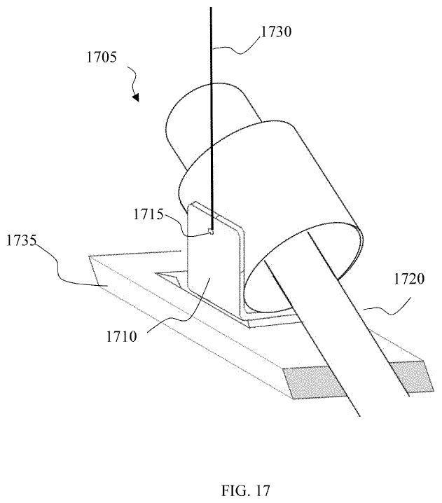

FIG. 17 shows a camera which can be attached to a speculum by using a camera adapter which allows the necessary leeway to tilt up the camera and reach an acute position upwards, according to exemplary embodiments of the present invention. FIG. 17 shows a camera 1705 connected to camera adapter 1710 designed to accommodate the camera 1705 and fix it in a stabilized fashion. Camera adapter 1710 can comprise a connecting platform 1735 designed to be connected to a medical device such as speculum. Camera adapter 1710 also comprises a hinge 1715 allowing to pivot and control the angle of the camera 1705. Such a control may be achieved by tilting the camera 1705 upwards and downwards. In some cases, the camera 1705 can be tilted upwards towards perpendicular axis 1730 and reach an acute angle between the camera 1705 and perpendicular axis 1730. Camera 1705 may also comprise a sleeve 1720 which in some cases can be an expendable sleeve. In some cases, sleeve 1720 may be a rigid sleeve which can be utilize to control the location of the camera 1705. In some embodiments of the present invention a user of the camera 1705 may utilize the sleeve 1720 to lock the position of the camera.

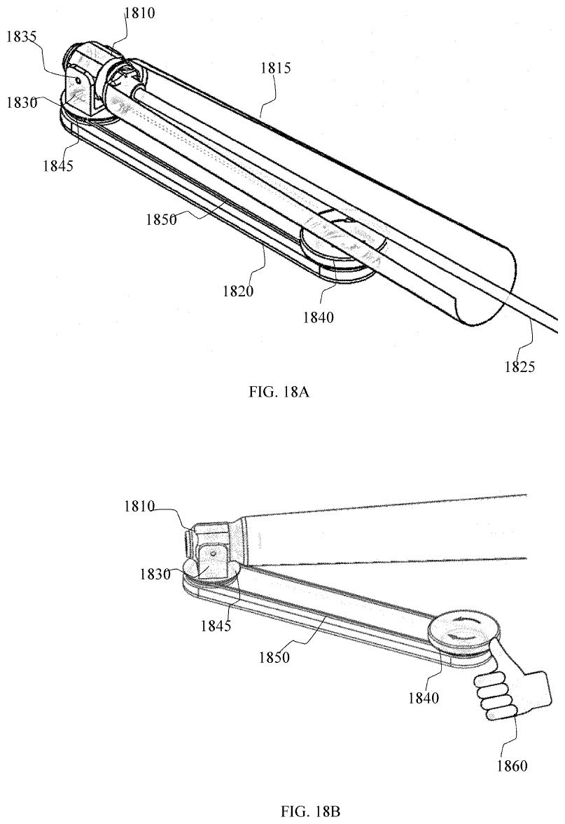

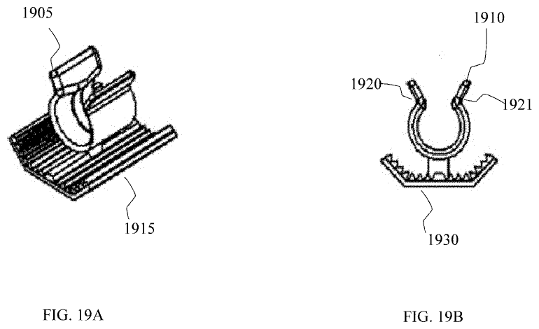

FIG. 18A shows a camera of an endoscope with a sleeve attached to a connecting platform which allows to pan the camera in the horizontal dimension, according to exemplary embodiments of the present invention. FIG. 18A shows a camera 1810 connected to camera adapter 1830 by hinge 1835 allowing to pivot and control the angle of the camera 1810. The camera adapter 1830 can be connected to a pan wheel 1845 which allows to rotate the camera 1810 right or left in the horizontal dimension. In some cases, the pan wheel 1845 may be connected to a connection platform 1820 designed to be attached to a speculum. For example, connection platform 1820 may be attached to the one of the blade members (not shown) of a speculum in a firm and steady fashion. Thus, the camera 1810 can be rotated to the right or to the left. The attachment of the connection platform 1820 to one of the blade members of a speculum may be by adding and adhesive material to the connection platform 1820. In some cases, a screws or magnets may be utilized to attach the connection platform 1820 to the blade member of the speculum. In some embodiments of the present invention a steering wheel 1840 may also be attached to the connection platform 1820 and connected with steering cable 1850. The steering wheel 1840 may be designed to be rotated clockwise and counterclockwise. Steering wheel 1840 may be connected to the pan wheel 1845 by the steering cable 1850. Thus, in case steering wheel 1840 is being rotated clockwise, the pan wheel 1840 may also rotate clockwise and the camera 1810 may move to point to the right. Similarly, in case steering wheel 1850 is being rotated counterclockwise, the pan wheel 1845 may also rotate counterclockwise and the camera 1810 may move to point to the left.

FIG. 18A also shows an expendable sleeve 1815 utilized to cover cables and wires the camera 1825. For example, the camera 1825 may be a video cable or a power cable connected to the camera 1810. Thus, the cables and the wires can be covered by the expendable sleeve 1815. In some cases, the expendable sleeve 1815 may also be utilized to control the upwards and downwards movements of the camera 1810. For example, in case a user wishes to change the position of the camera 1810, or change the direction to which camera 1810 is pointing, the user may utilize the expendable sleeve 1815 to tilt the camera 1810.

FIG. 18B demonstrates the operation changing the camera of an endoscope in the horizontal dimension by using a pan wheel and a steering wheel, according to exemplary embodiments of the present invention. FIG. 18B shows a camera 1810 connected to camera adapter 1830 by hinge 1835 allowing to pivot and control the angle of the camera 1810. The camera adapter 1830 can be connected to a pan wheel 1845 which allows to rotate the camera 1810 right or left in the horizontal dimension. FIG. 18B also shows a steering wheel 1840 connected with a steering cable 1850 to the pan wheel 1845. FIG. 18B shows the operation of the steering wheel to pivot the camera 1810 and thereby to change the pointing direction of the camera. FIG. 18B shows a hand 1860 which can rotate the steering wheel 1840 right or left, and thus to pivot the camera 1810 accordingly.

FIGS. 19A-19B show a flexible gripper designed to connect a camera of an endoscope to a speculum and provide the ability to change the direction to which the camera is pointing to, according to exemplary embodiments of the present invention. FIG. 19A shows a side view of flexible gripper 1905 which allows inserting of an endoscope camera. The flexible gripper 1905 can house the camera (not shown) and hold it in a steady and firm fashion. In some cases, the flexible gripper 1905 may allow moving the camera and change the direction to which the camera is pointing. Such changes may be on the horizontal dimensions or in some cases upwards or downwards in the vertical dimension. Flexible gripper 1905 can be connected to connection platform 1915 which can be attached to one of the blade members of a speculum. FIG. 19B also shows a front view of a flexible gripper 1910 attached to connection platform 1930. The flexible gripper may comprise side member 1920 and side member 1921 designed to clutch the endoscope camera (not shown) and hold the camera in a steady and firmed fashion.

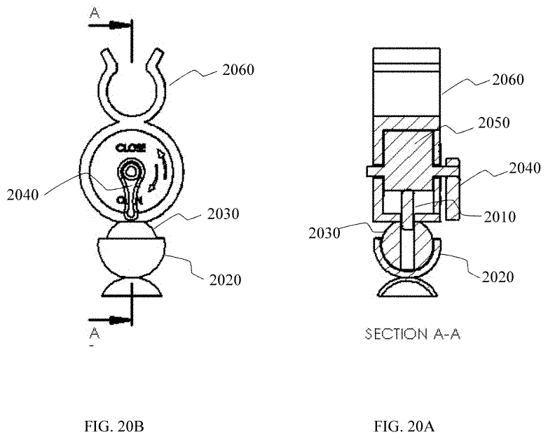

FIGS. 20A-20B show a flexible gripper and a locking mechanism of an endoscope of a speculum to a specific direction, according to exemplary embodiments of the present invention. The flexible gripper 2060 is attached to the body of the locking mechanism. A handle 2040 is configured to enable a user of the speculum to lock and unlock the endoscope in the speculum. The handle 2040 is connected to an eccentric tubular member 2050, such that moving the handle 2040 results in movement of the eccentric tubular member 2050. The eccentric tubular member 2050 is rigidly attached to a pin, for example a pin is attached to a single point in the eccentric tubular member 2050. Thus, moving the eccentric tubular member 2050 according to movement of the handle 2040 maneuvers the pin 2010. Locking the endoscope comprises moving a pin 2010 from one position to another. For example, the pin 2010 is in operating communication with a ball 2030 mounted on a cradle 2020. The cradle 2020 is mounted on top of a Velcro sticker for stabilizing the speculum apparatus. The ball 2030 may comprise a spring in a void in which the pin penetrates according to the movement of the handle 2040. Thus, when the spring is pressed, the endoscope is locked.

While the disclosure has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings without departing from the essential scope thereof. Therefore, it is intended that the disclosed subject matter not be limited to the particular embodiment disclosed as the best mode contemplated for carrying out this invention, but only by the claims that follow.

1. A speculum device, comprising:
a body, comprising a lower blade member and an upper blade member;
an imaging device for capturing images;
an adapter connected to the body, said adapter is connected to the camera and enables a user of the speculum device to adjust the field of view of the camera relative to the body.

2. The speculum device of claim 1, wherein the adapter has a spherical form which allows the person utilizing the device to adjust the position of the imaging device by making panning operations, tilting operations or rotate the camera.

3. The speculum device of claim 1, wherein the adapter further comprises an elongated hollow guide, wherein the camera is inserted into the device via the guide.

4. The speculum device of claim 3, wherein the guide tube comprises a cone shaped entrance designed to facilitate the insertion of the camera into the guide tube.

5. The speculum device of claim 1, further comprises a lock utilized for fixation of the camera relative to the two blade members, such that an angle between the camera field of view and the blade members is fixed and remains constant.

6. The speculum device of claim 1, wherein the adapter is a detachable adapter designed to be attached and/or detached from the speculum device.

7. The speculum device of claim 1, wherein the adapter is attached to the speculum via dedicated tiny rails placed in one of the speculum's blades, thus enabling movement of the camera along the blades to adjust the zoom of the camera.

8. The speculum device of claim 7, wherein the tiny rails are configured to host the adapter between two teeth on the rail and thereby fix the adapter and the camera movement.

9. The speculum device of claim 1, wherein the adapter is connected to the lower blade member.

10. The speculum device of claim 1, wherein the adapter is connected to the upper blade member.

11. The speculum device of claim 1, wherein the adapter comprises a round shaped casing and a spherical joint attached to the round shaped casing, wherein the camera is positioned in the round shaped casing.

12. The speculum device of claim 11, wherein the round shaped casing houses an eccentric socket, wherein the adapter further comprises a plurality of rods being in communication with the in the spherical joints, wherein the rods apply pressure on the spherical joints to lock the endoscope movement inside the round shaped casing.

13. The speculum device of claim 1, wherein the adapter also comprises a locking tooth configured to lock the camera inside the camera adapter.

14. The speculum device of claim 1, further comprises a hinge connecting the camera to the camera adapter, said hinge allows to pivot and control the angle of the camera relative to the blades.

15. The speculum device of claim 1, wherein the adapter further comprises a connecting platform connected to the speculum, said connecting platform is further connected to the camera.

16. The speculum device of claim 1, wherein the adapter also comprises a jagged aperture configured to fasten the camera in a specific location.

17. The speculum device of claim 16, wherein the jagged aperture is utilized to change the location of the camera without removing or changing the location of the adapter.

18. The speculum device of claim 1, wherein the adapter is connected to a pan wheel which allows to rotate the camera right or left in the horizontal dimension.

19. The speculum device of claim 18, wherein the pan wheel is connected to a connection platform attached to one of the blade members of the speculum.

20. The speculum device of claim 18, wherein the pan wheel is positioned between the camera and the connecting platform, further comprises a steering wheel mounted on a proximal section of the connecting platform, and a connecting cable connecting the pan wheel and the steering wheel, such that movement of the steering wheel results in movement of the pan wheel and thus rotation of the camera.

21. The speculum device of claim 1, further comprises a flexible gripper designed to connect a camera of an endoscope to a speculum, flexible gripper may comprise side member and side member designed to clutch the endoscope camera.

22. A medical gynecology examination method, comprising:
mounting a camera on a speculum device using an adapter, said adapter enables adjusting a pan of the camera relative to a blade of the speculum device;
inserting at least a portion of the speculum device into a patient's body; maneuvering the camera from a first position to a second position, the second direction has an angle relative to the blade of the speculum device that differs from the angle of the first direction;
capturing an image of the patient's body when the camera is in the second position.

23. The method according to claim 22, further comprises fixating the camera in the second direction.

24. The method according to claim 22, further comprises transmitting the captured image to a digital system, said digital system is configured to document the gynecology examination.
